(19) United States
(12) Patent Application Publication
(10) Pub. No.: US 2021/0121229 A1
(43) Pub. Date: Apr. 29, 2021

(54) IMPROVED DEVICE FOR ABLATION

(71) Applicant: Glenn VAN LANGENHOVE, Merelbeke (BE)

(72) Inventor: Glenn VAN LANGENHOVE, Merelbeke (BE)

(21) Appl. No.: 16/605,858

(22) PCT Filed: Apr. 18, 2017
(86) PCT No.: PCT/IB2017/000732

§ 371 Date: Oct. 17, 2019

(51) Int. Cl. A61B 18/14; A61B 18/02; A61B 18/24

(52) U.S. Cl. CPC A61B 18/1492; A61B 2018/00577; A61B 18/24; A61B 18/02

(57) ABSTRACT

The current invention concerns a catheterization system For performing an Interventional ablation procedure comprising: a flexible guiding catheter comprising a distal end for Insertion and a proximal end for manipulation, the distal end of the guiding catheter transformable from a substantially elongated shape for passage within the vasculature into a contact shape for contacting internal tissue along a continuous band, the flexible guiding catheter comprising a guiding lumen, and a flexible ablation catheter comprising an ablation tip near a distal end, the ablation catheter insertable or inserted into the guiding lumen of the guiding catheter, characterized In that the guiding catheter comprises a continuous slit at or near the distal end of the guiding catheter, said silt arranged such that, when the distal end of the guiding catheter is in said contact shape and contacts internal tissue along a continuous band, the silt defines a functional opening between the guiding lumen and the internal tissue along said continuous band, through which slit said ablation tip is capable of ablating said internal tissue along said continuous band. The Invention further concerns a guiding catheter and an interventional ablation procedure.

### TECHNICAL FIELD

The invention pertains to the technical field of surgical tools and methods for ablating tissue, in particular internal tissue, and more in particular ablating the Inner wall of a vessel or organ. The invention hereto concerns tools and methods which allow to ablate a continuous band on the tissue, in particular a continuous circular, spiraling, helical and/or substantially circumferential band. The tools and methods of the invention are particularly adept at treating atrial fibrillation by creating a circumferential ablation band on the Inner wall of the pulmonary vein, thereby obtaining pulmonary vein isolation. However, the tools and methods of the Invention can also be used for other similar treatments where a continuous band on internal tissue needs to be ablated.

### BACKGROUND

Atrial fibrillation (AP) is an arrhythmia of the heart causing Irregular electrical activity, followed by disorganized and ineffective contractions. It is the most common serious abnormal heart rhythm. AF can arise when electrical signals, typically travelling from a pulmonary vein (PV) towards the left atrium, trigger the heart cells of the left atrium, resulting in a discharge of these cells which is out of phase with the normal heart-beating cycle.

A recently developed treatment of AF is pulmonary vein Isolation (PVI), This procedure involves blocking the electrical paths between one or more pulmonary veins and the left atrium by ablation of the Inner wall of the PV, typically at the level of the antrum. The ablation causes scar tissue, which is non-conductive. For a good PVI, it is necessary to ensure that no signals car travel down the PV towards the atrium, and thus it is necessary to ablate an essentially circumferential band. If the ablated band is not circumferential, the risk that electrical signals travel down the PV to the left atrium remains high, i.e. the better one is capable of ablating a circumferential band, the lower the risk of AF recurrence after the ablation procedure.

The classic PVI procedure Involves Inserting an ablation catheter in the femoral vein, all the way to the right atrium, puncturing the interatrial septum, to be able to reach the antrium of the PV with the ablation tip of the catheter. This ablation tip can be a tip or, more typically, have the shape of a horseshoe. The circumferential band is then ablated by subsequent pressing of the ablation tip against the inner wall of the PV, hereby trying to ensure that a circumferential band is ablated.

However, the classic PVI procedure has the disadvantage that it does not always lead to a circumferential band, and that a second, third, or further, procedure needs to be performed. It is clear that this is undesirable.

An alternative method, which limits the surgical procedure drastically, is disclosed in WO 2012/131107 A1, which discloses systems, devices and methods for the ablation of a vessel's wall from the inside, more specifically to implant devices and to the ablation of the wall of one or more pulmonary veins (PV) from the inside, preferably transmural ablation and preferably at the level of the antrum. Hereby, one or more implant devices can be implanted in the vessels and can subsequently be heated by external energy-providing means.

Other conditions exist which can be or could be treated by ablating signal blocking bands, such as atrial tachycardia, atrial flutter, ventricular tachycardia, or other focal arrhythmias, and further arterial hypertension, norepinephrine spillover, heart failure, hypertension related target organ damage, etc.

Document U.S. Pat. No. 5,873,855 A discloses a spiral catheter apparatus for access to, and laser or other treatment within, cavities and organs in the human body, the apparatus comprising a flexible, main catheter shaft defining a central axis of the apparatus, the catheter shaft having a proximal end, a distal end and a first hollow lumen region extending therethrough, the catheter shaft further having a spiral portion adjacent the distal end with a selected curvilinear shape, the curvilinear shape defining an inner arcuate sidewall and an outer arcuate sidewall, the catheter shaft flexible enough to assume a temporarily elongated shape such that the apparatus can be extended through at least a portion of the body In the temporarily elongated shape and will assume the selected curvilinear shape when extended Into a body cavity or organ, the selected curvilinear shape serving to securely position the apparatus adjacent a selected surface within the body cavity or organ, at least the spiral portion of the catheter shaft having a plurality of guide holes thereon, the plurality of guide holes disposed at least on the outer arcuate sidewall in communication with the first lumen such that a distal end of a laser delivery means or other functional device can be controllably advanced through the plurality of guide holes for laser or other treatment on the selected surface. The catheter of U.S. Pat. No. 5,873,685 is unsuitable for ablating a continuous band on the tissue.

International patent application WO 98/22176 A1 discloses a guiding introducer containing openings which is used with an ablation catheter of cardiac tissue. The introducer comprises a first proximal section, generally elongated and hollow, and a second distal section, lumened with a plurality of openings. The openings have a sufficient size to permit ablation of cardiac tissue through them by an electrode of the ablation catheter. The catheter allows point-per-point ablation by an ablation electrode through the distinct openings. However, such a catheter is not configured to achieve a continuous band and also cannot ensure a continuous band to be ablated.

Furthermore, the ablation catheter is larger than the openings and is thus clearly not meant to be In direct contact with the tissue, thereby limiting the possibility of ablation by heat transfer.

There remains a need In the art for Improved devices, systems and methods for ablating continuous bands on internal tissue, in particular on Inner walls of vessels and organs. In many applications, the continuous band need to form an essentially closed loop, e.g. an essentially circumferential band on a vessel's inner wall, or an essentially circular band on an organ's inner or outer wall around an ostium.

There also remains a need in the art for improved devices, systems and methods which allow the ablation of continuous bands in a short period. Typically during the ablation procedure, monitoring of the position of the catheters is performed with fluoroscopy. By shortening the procedure, the patient's body is less exposed to radiation.

The shape of vessels and organs can differ significantly from patient to patient. Hence, there also remains a need for devices, systems and methods, wherein the continuous bands can be ablated on internal tissue of vessels or walls of varying shape and size. Hereby, preferably, the devices offer a flexibility which allows creating a continuous ablation, band on the internal tissue, for a multitude of shapes and sizes.

### SUMMARY OF THE INVENTION

The present Invention provides a catheterization system for performing an Interventional ablation procedure. The system comprises:
a flexible guiding catheter comprising a distal end fur Insertion and a proximal end for manipulation, the distal end of the guiding catheter transformable from a substantially elongated shape for passage within the vasculature into a contact shape for contacting Internal tissue along a continuous band, the flexible guiding catheter comprising a guiding lumen, and
a flexible ablation catheter comprising an ablation tip near a distal end, the ablation catheter insertable or inserted into the guiding lumen of the guiding catheter, characterized in that the guiding catheter comprises a continuous slit at or near the distal end of the guiding catheter, said slit arranged such that, when the distal end of the guiding catheter is in said contact shape and contacts internal tissue along a continuous band, the slit defines a functional opening between the guiding lumen and the internal tissue along said continuous band, through which slit said ablation tip is capable of ablating said internal tissue along said continuous band.

Flexible catheters are typically used in interventional procedures. The system of the present Invention comprises at least two catheters, one used for guiding and/or positioning, and another used for performing the ablation. The shape of the distal end of the guiding catheter can be changed from a substantially elongated shape to a contact shape. The elongated shape can be applied when the catheter is being Inserted Into the vasculature of a patient, either directly or within another catheter. The contact shape can be applied when the distal end of the guiding catheter is essentially at the position of where the ablation procedure needs to take place, e.g. in the antrum of a pulmonary vein or in the left atrium at or hear a pulmonary vein If the ablation procedure consists of pulmonary vein isolation. Depending on the exact procedure and on the specifics of the vessel's or organ's wall which needs to be treated, the contact shape may be made to vary. The contact shape preferably is spiraling, helical or essentially circular, elliptical or cylindrical.

In the present invention, the interventional ablation procedure concerns a process in which a continuous band needs to be ablated, rather than local, point-like zones. An ablation along a continuous band is typically necessary in procedures intended to create lesions which block electrical signals. Such electrical signals may travel along specific paths, e.g. along nerves on the vessel or organ, the exact position of the paths not always known or varying from patient to patient. In these cases, ablation needs to be performed over an extended area, i.e. a continuous band, in order to ascertain that the signal-conducting path is being blocked. Furthermore, the ablated path preferably forms an essentially closed curve, such as an essentially complete circumferential path, such essentially closed curves defining at least two regions of the vessel or organ which are intended to be electrically isolated from each other.

In order to be able to perform the ablation over a continuous band, a continuous silk is provided at or near the distal end of the guiding catheter, said slit arranged such that, when the distal end of the guiding catheter is in said contact shape and contacts internal tissue along a continuous band, the slit defines a functional opening between the guiding lumen and the internal tissue along said continuous band, through which slit said ablation tip is capable of ablating said Internal tissue along said continuous band. Hence, the system of the present invention is better capable, of ablating a continuous band, preferably an essentially helical, spiral and/or circumferential band. This is achieved by inserting the ablation catheter through the guiding lumen of the guiding catheter, and by ablating the targeted tissue with the ablation catheter through the slit. A continuous ablated band can be achieved by moving the ablating tip of the ablation catheter during ablation along at least part of said slit, and preferably along the entire slit. Alternatively, the ablation tip may comprise an ablative tip portion extending over a pre-determined length of the ablation catheter, said pre-determined length being determined on the basis of the length of the continuous band which needs to be ablated. Hence, the system of the present invention allows better ablation than the conventional techniques because it separates the functions of positioning of the catheters and ablating of the tissue, thereby better controlling each aspect, i.e. positioning can be better controlled due to the distal end of the guiding catheter which can be dedicated to providing the best contact shape, thereby ensuring that the silk is positioned In functional contact with the targeted ablation zone, and ablation can be better controlled due to the separate control of the ablating catheter during ablation. Furthermore, the type of ablation tip and ablation process can also be selected more freely, i.e. in function of the best expected ablation results, and not necessarily in function of the geometry of the treated vessel or organ.

The present invention also provides in a flexible guiding catheter comprising a distal end for insertion and a proximal end for manipulation, the distal end of the guiding catheter transformable from a substantially elongated shape for passage within the vasculature into a contact shape for contacting internal tissue along a continuous band, the flexible guiding catheter comprising a guiding lumen, wherein the guiding catheter comprises a continuous slit at or near the distal end of the guiding catheter, said slit arranged such that, when the distal end of the guiding catheter is In said contact shape and contacts Internal tissue along a continuous band, the slit defines a functional opening between the guiding lumen and the internal tissue along said continuous band, through which slit said ablation tip is capable of ablating said internal tissue along said continuous band.

The present invention also provides in an interventional ablation procedure. This method comprises the steps of:
inserting a flexible guiding catheter into vasculature of a patient, said guiding catheter comprising a guiding lumen and a continuous slit at or near a distal end of the guiding catheter;
positioning the distal end of said guiding catheter at an ablation treatment zone and transforming the distal end of said guiding catheter into a contact shape, thereby contacting internal tissue along a continuous band, whereby said slit provides a functional opening between the guiding lumen and the internal tissue along said continuous band;
inserting a flexible ablation catheter into the guiding lumen of said guiding catheter;
ablating the internal tissue along said continuous band with said ablation catheter via said silt.

In an embodiment, the method is followed by a step of rotating the guiding catheter while ablating, preferably by proximally manipulating the guiding catheter. Such a rotation is preferably performed over an angle of at least 1°, more preferably at least 5° and even more preferably at least 10°, and preferably at most 90°, more preferably at most 75°, still more preferably at most 60°, yet more preferably at most 45°, most preferably between 10° and 45° such as 11°, 12°, 13°, 14°, 15°, 16°, 17°, 18°, 19°, 20°, 21°, 22°, 23°, 24°, 25°, 26°, 27°,'28°, 29°, 30°, 31°, 32°, 33°, 34°, 35°, 36°, 37°, 38°, 39°, 40°, 41°, 42°, 43°, 44°, or any value therebetween. Performing such a step may ensure that a complete circumferential lesion is obtained, even in the case the contact shape is a spiral, a helical shape or an incomplete circular shape. Although the present invention already provides near certainty that a circumferential lesion can be obtained without performing the extra rotating step, the extra rotating step may be deemed necessary, especially in the case of uncertainties with respect to e.g. anatomical variations such as uncommonly shaped vessels, e.g. pulmonary veins.

Alternatively, or additionally, the guiding catheter comprises an opening, such as a local widening of the slit near a distal end or proximal end of the silt, through which the ablation catheter can be extended, in particular further than through the other portions of the slit.

### DESCRIPTION OF FIGURES

FIGS. 1 to 6 show an ablation catheter according to the present invention from different viewpoints.

FIG. 7 shows a further embodiment of the present invention.

### DETAILED DESCRIPTION or THE INVENTION

The present invention concerns a catheterization system for performing an Interventional ablation procedure and an interventional ablation procedure according to the claims and as further specified In this document.

Unless otherwise defined, all terms used in disclosing the invention, including technical and scientific terms, have the meaning as commonly understood by one of ordinary skill in the art to which this invention belongs. By means of further guidance, term definitions are Included to better appreciate the teaching of the present invention. As used herein, the following terms have the following meanings:

“A”, “an”, and “the” as used herein refers to both singular and plural referents unless the context clearly dictates otherwise, By way of example, “a compartment” refers to one or more than one compartment.

“About” as used herein referring to a measurable value such as a parameter, an amount, a temporal duration, and the like, is meant to encompass variations of +/−20% or less, preferably +/−1.0% or less, more preferably +/−5% or less, even more preferably +/−1% or less, and still more preferably +/−0.1% or less of and from the specified value, in so far such variations are appropriate to perform in the disclosed Invention. However, it is to be understood that the value to which the modifier “about” refers is Itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known In the art or disclosed therein.

The term “rail” or “gutter” as used herein refers to a portion, preferably located at or near the distal end, of a guiding catheter comprising a slit, the portion being arranged to guide another catheter, e.g. an ablation catheter or a sensing catheter, along the slit.

The recitation of numerical ranges by endpoints includes all numbers and fractions subsumed within that range, as well as the recited endpoints.

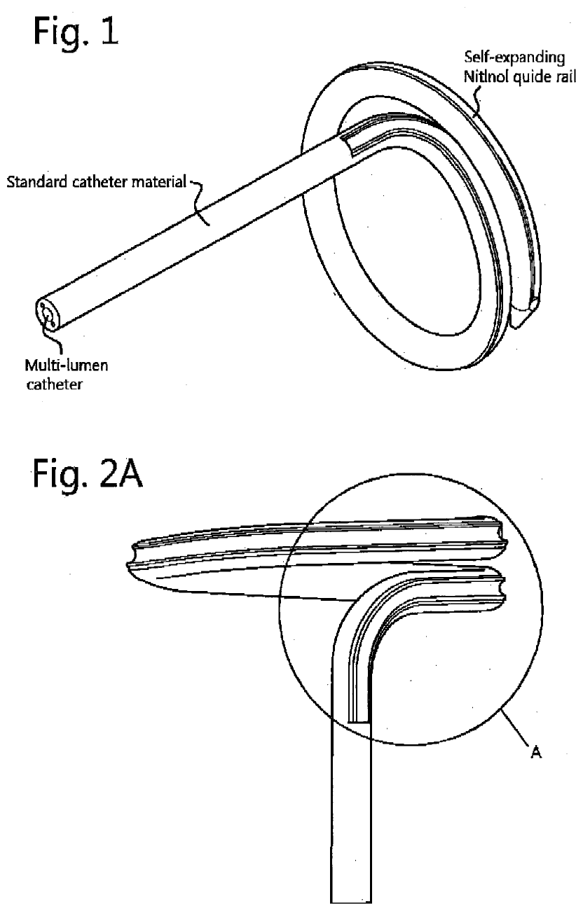

In a first aspect, the invention provides catheterization system for performing an interventional ablation procedure. The system comprises a guiding catheter and an ablation catheter. A guiding catheter according to the present invention is shown in FIGS. 1 to 4.

In a preferred embodiment, the guiding catheter comprises a self-expanding distal end, which is transformable from a substantially elongated shape for passage within the vasculature into a contact shape for contacting internal tissue along a continuous band. In a preferred embodiment, the distal end comprises nitinol for allowing the transforming of the distal end in a self-expanding manner. Note that the self-expanding distal end is responsible for transforming the shape of the guiding catheter into the contacting shape, which preferably comprises a helical shape, a spiral shape or an essentially circular shape, but that the diameter of the guiding catheter is essentially fixed. The self-expanding distal end can hereby transform by curling up into such a preferred helical shape, spiral shape or circular shape, thereby leaving the diameter of the guiding catheter unchanged. A particularly preferred embodiment thus comprises a guiding catheter in which the distal end is provided with a nitinol wire or nitinol structure along at least a portion of the distal end of the guiding catheter, preferably provided essentially in a diametrically opposite half of the guiding catheter with respect to the slit, wherein the nitinol wire or structure comprises an unexpanded state In which it is substantially elongated for passage within the vasculature, and a deployed state defining the contact shape of the distal end of the guiding catheter, preferably the contact shape comprising a helical shape, spiral shape or circular shape. In an embodiment, the guiding catheter and/or the ablating catheter, and optionally all additional catheters such as sensing catheters, are steerable and/or deflectable. Steerable and deflectable catheters allow rotation and/or deflection resp. of at least the distal end of a catheter. Further, the catheters of the present invention may be steerable fixed-curve, bi-directional, 4-way deflectable, uni-directional or omnidirectional.

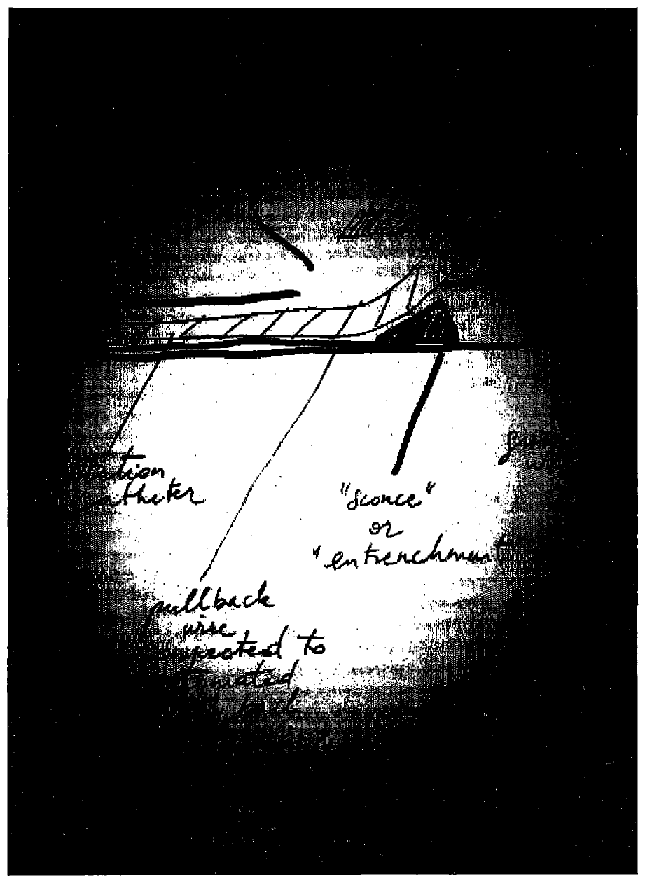

In a preferred embodiment, the catheterization system of the present invention may comprise a catheter-in-guide wherein at least one catheter is telescopically applicable or applied in another catheter. In FIG. 6, a lengthwise cross section of a catheterization system is shown which comprises a catheter-In-guide. In the shown embodiment, the ablation catheter telescopically fits an ablation positioning catheter, which can be inserted Into the guiding lumen of the guiding catheter either separately or simultaneously with the ablation catheter. The ablation positioning catheter comprises a distal end with an opening, the opening allowing the passage of at least the ablation tip of the ablation catheter, preferably such that the ablation tip can stick out of the slit in the guiding catheter and can come Into direct contact with the internal tissue during ablation. This cart be achieved by a guiding stop at the distal end of the ablation positioning catheter, which can guide the distal end of the ablation catheter with the ablation tip outwards, through the silk of the guiding catheter. With this embodiment, the length of the ablation catheter which sticks out of the silt, can be easily and accurately arranged proximally, which also allows an easy and accurate control of the pressure applied with the ablation tip to the internal tissue during the procedure. A continuous band can be ablated by essentially fixing the position of the ablation catheter with respect to the ablation positioning catheter in an ablative position, whereby the ablation tip sticks out of the slit and is in functional or direct contact with the internal tissue, and subsequently moving the ablation catheter and ablation positioning catheter along the lumen of the guiding catheter, such the ablation tip ablates a continuous band on the internal tissue as determined by the slit.

In preferred embodiments, the contact shape comprises a helical shape, a spiral shape or an essentially circular shape. In FIGS. 1 to 4, a contact shape comprising an essentially circular shape is Illustrated, a top view is presented in FIG. 4.

Preferably, the contact shape is selected taking into account the vessel or organ onto which the ablation is to be performed. In an embodiment, the contact shape may preferably be essentially circular. A circular shape can be preferred if a circumferential band needs to be ablated on the inner wall of a vessel, such as is the case for ablation in the antrum of a pulmonary vein to achieve PVI. A circular shape may also be preferred in case a surrounding band needs to be ablated on an organ wall around the entry or exit of a vessel, such as can be the case for ablation In the ostium of a pulmonary vein for achieving PVI, the ablation band thereby surrounding the entry of the pulmonary vein in the left atrium. In this latter case, a spiraling shape may also be preferred. A helical shape may be preferred if a helical band needs to be ablated on the inner wall of a vessel, such as is the case for ablation in a renal artery to treat arterial hypertension,

The silt comprises a length as measured along the guiding catheter, and a width as measured in an essentially azimuthal direction with respect to the guiding catheter. By definition, the slit is longer than it is wide. The width of the slit may vary along its length, but preferably the width of the silt is essentially constant along its length. Preferably, the slit comprises a length of at least 1 cm, more preferably at feast 1.5 cm, still more preferably at least 2 cm, yet more preferably at least 3 cm, at least 4 10 cm, at least 5 cm, at least 6 cm or even longer. Possible lengths of the slit are 1.0 cm, 1.1 cm, 1.2 cm, 1.3 cm, 1.4 cm, 1.5 cm, 1.6 cm, 1.7 cm, 1.8 cm, 1.9 cm, 2.0 cm, 2.1 cm, 2.2 cm, 2.3 cm, 2.4 cm, 2.5 cm, 2.6 cm, 2.7 cm, 2.8 cm, 2.9 cm, 3.0 cm, 3.1 cm, 3.2 cm, 3.3 cm, 3.4 cm, 3.5 cm, 3.6 cm, 3.7 cm, 3.8 cm, 3.9 cm, 4.0 cm, 4.1 cm, 4.2 cm, 4.3 cm, 4.4 cm, 4.5 cm, 4.6 cm, 4.7 cm, 4.8 cm, 4.9 cm, 5.0 cm, 5.1 cm, 5.2 cm, 5.3 cm, 5.4 cm, 5.5 cm, 5.6 cm, 5.7 cm, 5.8 cm, 5.9 cm; 6.0 cm, 6.1 cm, 6.2 cm, 6.3 cm, 6.4 cm, 6.5 cm, 6.6 cm, 6.7 cm, 6.8 cm, 6.9 cm, or any value there between or even higher than 7 cm. Preferably, the silt comprises a width which is smaller than 1 cm, more preferably smaller than 0.8 cm, still more preferably smaller than 0.5 cm, Possible widths of the slit are 1 cm, 0.9 cm, 0.8 cm, 0.7 cm, 0.6 cm, 0.5 cm, 0.4 cm, 0.3 cm, 0.2 cm, 0.1 cm or any value therebeteween or even smaller than 0.1 cm. In embodiments wherein a circumferential band needs to be ablated on the Inner wall of a vessel, such as is the case for ablation in the antrum of a pulmonary vein to achieve PVI, the slit comprises a length which is at least as long as the circumference of the of the vessel.

In an embodiment, the ablation catheter is a contact ablation catheter, i.e. it ablates tissue by direct contact of the ablation tip with the tissue. A contact ablation catheter can for instance be a heating catheter comprising an ablation tip which ablates tissue by direct heat transfer from ablation tip to tissue. In embodiments where the ablation catheter is a contact ablation catheter, the slit comprises a width which is larger than a width of the ablation tip of the contact ablation catheter in order to allow passage of at least a portion of the ablation tip through the silt.

In an alternative embodiment, the ablation catheter is a contactless catheter, i.e. it ablates tissue without direct contact of the ablation tip with the tissue. In such an embodiment, the slit may comprise a width which is larger than a width of the ablation tip of the contactless ablation catheter but preferably, the slit comprises a width which is smaller than a width of the ablation tip of the contactless ablation catheter

In related embodiments, it is the Intention to subsequently use more than one ablation catheter, e.g. a contact ablation catheter and, subsequently or prior, a contactless ablation catheter. This can be done using the same guiding catheter. The present invention thus relates to a system as described in this document, the system comprising a plurality of ablation catheters which are each insertable into the guiding lumen of the guiding catheter. The plurality of ablation catheters may comprise a contact ablation catheter and a contactless ablation catheter. In such embodiments, the ablation tip of the contact ablation catheter is smaller than a width of the slit and the ablation tip of the contactless ablation catheter is preferably larger than a width of the silt.

In a preferred embodiment, the guiding catheter comprises exactly one slit defining a functional opening between the guiding lumen and the internal tissue along the continuous band, through which slit the ablation tip is capable of ablating said internal tissue along the continuous band. In an alternative embodiment, the guiding catheter comprises a plurality of slits, e.g. two, three, four or more slits, provided at least partially adjacent to each other along the guiding catheter. Such an embodiment allows for instance a double continuous band to be ablated In a single procedure using one, two or more ablation catheters.

In an embodiment, the slit is located on the outward-lying side of the guiding catheter when the distal end of the guiding catheter has the contact shape (see FIG. 1). Such a silt allows essentially circumferential or partially circumferential bands to be ablated on the walls of a vessel. Hereby, the distal end of the guiding catheter in an elongated shape can be inserted into the vessel, subsequently the distal end of the guiding catheter can be transformed in an at least partially circumferential or helical contact shape. As the silt is located on the outward-lying side of the guiding catheter, the slit automatically faces the vessel's inner wall, which allows an easy and accurate subsequent ablation along an at least partially circumferential or helical band.

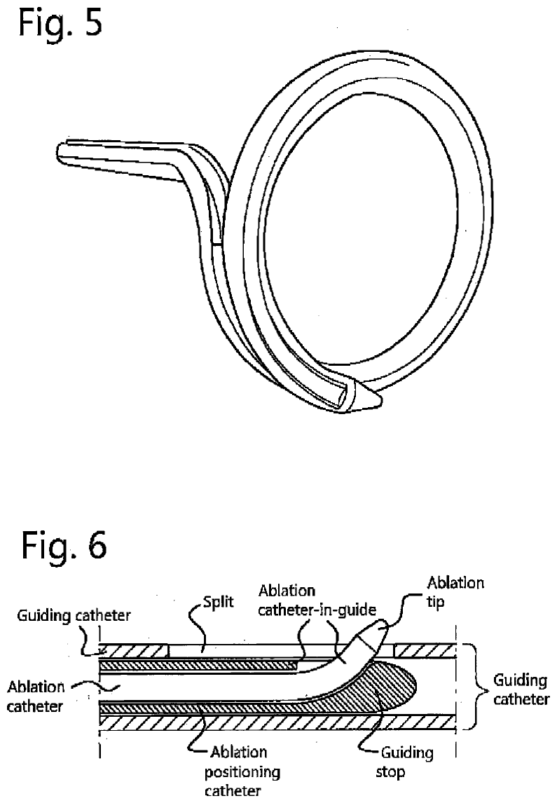

In another embodiment, the slit is located on the distal-lying side of the guiding catheter when the distal end of the guiding catheter has the contact shape (see FIG. 5). Such a slit allows essentially circular, spiraling, partially circular or partially spiraling bands to be ablated on the walls of an organ, preferably said band surrounding the entry or exit of a vessel into or out off said organ. Hereby, the distal end of the guiding catheter in an elongated shape can be inserted into the organ, subsequently the distal end of the guiding catheter can be transformed in an at least partially circular or spiraling contact shape. As the slit is located on the distal lying side of the guiding catheter, the slit automatically faces the organ's wall, preferably the silt at least partially surrounding the entry or exit of a vessel, e.g. the silt preferably facing the ostium of a vessel, which allows an easy and accurate subsequent ablation along an at feast partially circular or spiraling band.

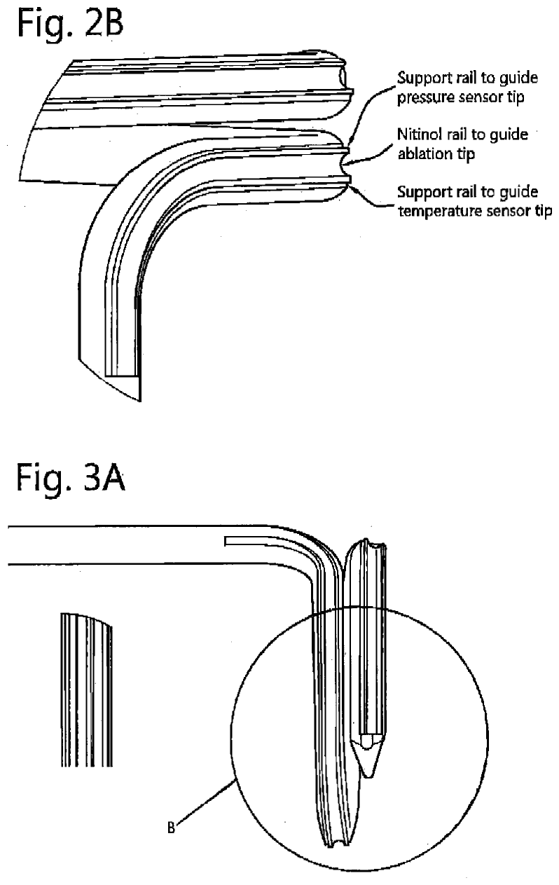

The guiding catheter comprises at least a guiding lumen. In a preferred embodiment, the guiding catheter comprises one, two or more additional lumens. These additional lumens may comprise a sensing slit at or near the distal end of the guiding catheter, said sensing slit arranged such that the slit defines a functional opening between an additional lumen and the Internal tissue. In FIGS. 2A and 2B, a guiding catheter with two additional lumens is shown in detail. These lumens preferably allow other catheters to be positioned with a distal end at or near the targeted region, preferably said other catheters comprising sensors. In a particularly preferred embodiment, the guiding catheter comprises a pressure sensing lumen and/or a temperature sensing lumen. Herein, the pressure sensing lumen allows to position a pressure sensing catheter with a distal end at or near the targeted ablation region and the temperature sensing lumen allows to positron a temperature sensing catheter with a distal end at or near the targeted ablation region.

In a preferred embodiment, the catheterization system comprises a sensing catheter for sensing a variable at or near the targeted ablation zone. In a particularly preferred embodiment, the system comprises a pressure sensing catheter and/or a temperature sensing catheter. Sensing catheters preferably comprise one or more sensors at or near a distal end of said sensing catheters, e.g. a pressure sensing catheter preferably comprises pressure sensor at its distal end and a temperature sensing catheter preferably comprises a temperature sensor at its distal end. These embodiments are preferably combined with a guiding catheter comprising one, two or more additional lumens as described here above. Sensing can be performed before, during and/or after the procedure.

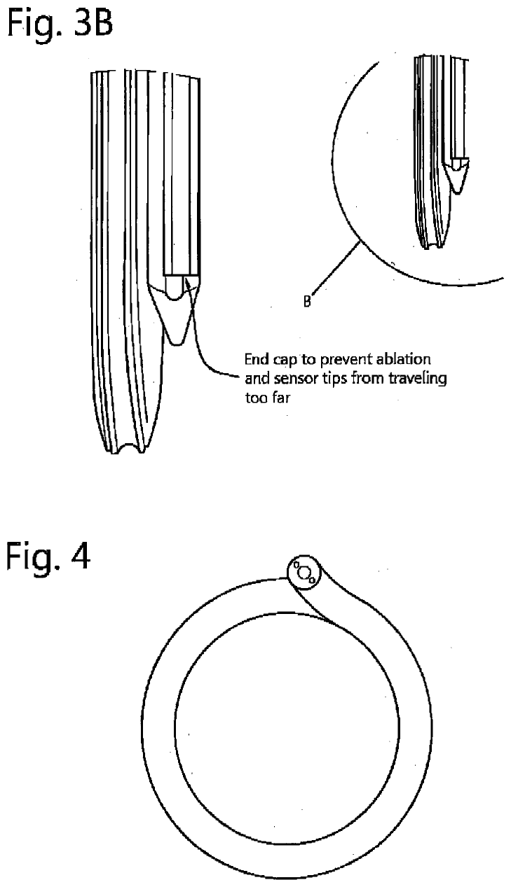

In a preferred embodiment, the distal end of the guiding catheter comprises an end cap closing off at least the guiding lumen and optionally the additional lumens, to prevent the ablation catheter and optionally sensing catheters from being inserted too far. This is shown in detail In FIGS. 3A and 3B.

In a preferred embodiment, the ablation catheter is a laser ablation catheter, an RF ablation catheter, a DC ablation catheter or a cryoablation catheter.

In a preferred embodiment of the invention, the guiding lumen comprises a cross section which is rotationally asymmetric around a longitudinal axis. Herein, the longitudinal axis refers to an axis oriented along the catheter, essentially through the geometrical center of the crass sections the lumen slang the catheter. For instance, the guiding lumen may have an essentially elliptical, triangular or rectangular cross section. Preferably in combination herewith, the ablation catheter preferably comprises a cross section which is rotationally asymmetric around a longitudinal axis, the rotational asymmetry being essentially of the same shape and size as the rotational asymmetry of the cross section or the guiding lumen. With such rotationally asymmetric cross sections, the ablation catheter is prevented to rotate within the guiding lumen during insertion into the guiding catheter, ensuring a better control of the ablation tip of the ablation catheter at the position of the distal end of the guiding catheter and thereby at the position of the silt. This embodiment is particularly preferred if the ablation catheter is a laser ablation catheter. In this case the ablation tip may comprise an outwardly oriented laser output region, i.e. a region where laser light is arranged to come out from the ablation tip In the direction of the slit towards the targeted tissue.

In an embodiment, the slit is open. In cases where the ablation catheter is arranged for ablating tissue by contacting the tissue with the ablation tip, such an open slit is necessary. Such can be the case for cryo-ablation, RF or DC ablation. In such cases it is also preferred that the ablation tip is of such dimensions that at least part of the ablation tip sticks out of the slit in order to contact the tissue, such as illustrated in FIG. 5.

In an alternative embodiment, the slit is at least partially dosed off fluidically, i.e. the slit does not or only partially allow fluid, e.g. blood, to be exchanged between the guiding lumen and the patient's body, e.g. the patient's vasculature. This embodiment can be preferred in cases where the ablation catheter is arranged contactless ablation, as can be the case for laser ablation or thermal ablation. For laser ablation in particular, the slit may be functionally open for the laser light by being transparent. For thermal ablation, the slit may be functionally open by comprising material of low thermal resistance.

In a preferred embodiment, at least one of the additional lumens is arranged for applying cooling liquid, preferably along the length of the guiding catheter, to cool the guiding catheter, and optionally the ablation catheter or other catheters, e.g. sensing catheters, during the procedure. The cooling liquid may also be used to cool the blood and/or vasculature along the guiding catheter during the Interventional procedure.

In a preferred embodiment, at least one of the additional lumens comprises one or more fluid openings at or near the distal end of the catheter and/or along, which fluidically connect the additional lumen to the vasculature for allowing transfer or exchange of fluids between vasculature and the additional lumen. Such additional lumen may be used for Introducing fluids Into the vasculature far treatment purposes, e.g. fluids comprising active substances, or cooling purposes, e.g. for cooling down the blood or the internal tissue or vasculature.

In an embodiment, the proximal end of the ablation catheter is directly or indirectly connected to an engine for moving the ablation catheter along the guiding lumen of the guiding catheter. Such an engine allows to better control the movement of the ablation catheter and thereby also the ablation tip. In particular during ablation of the internal tissue. Preferably, the ablation catheter can be rimed at an essentially constant speed, thereby allowing the achievement of a very regular ablation over the entire ablation band.

A further embodiment of the present invention is shown in FIG. 7. Here the catheter system comprises a sconce or entrenchment device which is proximally manipulable, preferably by a pullback wire which is preferably connected to a pullback system. The entrenchment device is preferably positioned near the distal end of the slit in the beginning of the procedure and comprises a slanted portion with respect to the longitudinal direction of the guiding catheter, such that an ablation catheter's tip is forced outwards towards and preferably through the slit when It is pushed distally. The entrenchment can be pulled back while the ablation catheter is not such that the ablation catheter's tip is pushed further outwards, or the entrenchment can be pushed without moving the ablation catheter such that the ablation tip is retracted inwards. Alternatively, ablation catheter and entrenchment device can be manipulated similarly such that the ablation is performed along the slit with the distance between the ablation tip and the guiding catheter's outer wall being the same during the procedure.

1. Catheterization system for performing an interventional ablation procedure comprising:
a flexible guiding catheter comprising a distal end for insertion and a proximal end for manipulation, the distal end of the guiding catheter transformable from a substantially elongated shape for passage within the vasculature into a contact shape for contacting internal tissue along a continuous band, the flexible guiding catheter comprising a guiding lumen, and
a flexible ablation catheter comprising an ablation tip near a distal end, the ablation catheter insertable or inserted into the guiding lumen of the guiding catheter,
characterized in that the guiding catheter comprises a continuous slit at or near the distal end of the guiding catheter, said slit arranged such that, when the distal end of the guiding catheter is in said contact shape and contacts internal tissue along a continuous band, the slit defines a functional opening between the guiding lumen and the internal tissue along said continuous band, through which slit said ablation tip is capable of ablating said internal tissue along said continuous band.

2. The catheterization system according to claim 1, wherein the guiding catheter comprises a self-expanding distal end, which is transformable from a substantially elongated shape for passage within the vasculature into a contact shape for contacting internal tissue along a continuous band.

3. The catheterization system according to claim 1, wherein the contact shape is a circumferential contact shape.

4. The catheterization system according to claim 1, wherein the contact shape comprises a helical shape, a spiral shape or an essentially circular shape.

5. The catheterization system according to claim 1, wherein the guiding catheter comprises one, two or more additional lumens.

6. The catheterization system according to claim 5, wherein at least one of the additional lumens comprises a sensing slit at or near the distal end of the guiding catheter, said sensing slit arranged such that the slit defines a functional opening between the additional lumen and the internal tissue.

7. The catheterization system according to claim 1, comprising a sensing catheter for sensing a variable at or near the targeted ablation zone.

8. The catheterization system according to claim 5, comprising a temperature sensing catheter.

9. The catheterization system according to claim 5, comprising a pressure sensing catheter.

10. The catheterization system according to claim 1, wherein the ablation catheter is a laser ablation catheter, an RF ablation catheter, a DC ablation catheter or a cryoablation catheter.

11. The catheterization system according to claim 1, wherein the guiding lumen comprises a cross section which is rotationally asymmetric around a longitudinal axis of the guiding lumen and wherein the ablation catheter comprises a cross section which is rotationally asymmetric around a longitudinal axis.

12. The catheterization system according to claim 1, wherein the ablation catheter telescopically fits an ablation positioning catheter, which comprises a distal end with an opening, the opening allowing the passage of at least the ablation tip of the ablation catheter.

13. A flexible guiding catheter comprising a distal end for insertion and a proximal end for manipulation, the distal end of the guiding catheter transformable from a substantially elongated shape for passage within the vasculature into a contact shape for contacting internal tissue along a continuous band, the flexible guiding catheter comprising a guiding lumen, and
characterized in that the guiding catheter comprises a continuous slit at or near the distal end of the guiding catheter, said slit arranged such that, when the distal end of the guiding catheter is in said contact shape and contacts internal tissue along a continuous band, the slit defines a functional opening between the guiding lumen and the internal tissue along said continuous band, through which slit said ablation tip is capable of ablating said internal tissue along said continuous band.

14. Interventional ablation procedure comprising the steps of:
inserting a flexible guiding catheter into vasculature of a patient, said guiding catheter comprising a guiding lumen and a continuous slit at or near a distal end of the guiding catheter;
positioning the distal end of said guiding catheter at an ablation treatment zone and transforming the distal end of said guiding catheter into a contact shape, thereby contacting internal tissue along a continuous band, whereby said slit provides a functional opening between the guiding lumen and the internal tissue along said continuous band;
inserting a flexible ablation catheter into the guiding lumen of said guiding catheter;
ablating the internal tissue along said continuous band with said ablation catheter via said slit.

15. The catheterization system according to claim 2, wherein distal end comprises nitinol for allowing the transforming of the distal end in a self-expanding manner.

16. The catheterization system according to claim 11, wherein the rotational asymmetry of the ablation catheter is essentially of the same shape and size as the rotational asymmetry of the cross section of the guiding lumen.

17. The catheterization system according to claim 12, wherein the ablation tip can stick out of the slit in the guiding catheter and can come into direct contact with the internal tissue during ablation.
